(19) United States
(12) Patent Application Publication
(10) Pub. No.: US 2008/0118555 A1
(43) Pub. Date: May 22, 2008

(54) Stable pharmaceutical composition containing desloratadine

(75) Inventors: Anandi Krishnan, Navi Mumbai (IN); Arra Ganga Srinivas, Navi Mumbai (IN); Deepika V. Bhat Tamragouri, Kalwa (W) Thane (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/584,287

(22) Filed: Oct. 20, 2006

Related U.S. Application Data
(60) Provisional application No. 60/801,683, filed on May 19, 2006.

(30) Foreign Application Priority Data
Oct. 20, 2005 (IN) 1320/MUM/2005

(51) Int. Cl. A61K 31/473; A61K 9/48; A61K 9/20

(52) U.S. Cl. 424/452; 514/290; 424/465

(57) ABSTRACT

Stable pharmaceutical compositions for oral administration are provided comprising an anti-allergic effective amount of descarbonylethoxy-loratadine or a pharmaceutically acceptable salt thereof, a lactose-based excipient and a pharmaceutically acceptable filler wherein the composition is substantially free of a pharmaceutically acceptable basic salt and wherein the composition contains less than about 1% by weight of N-formyl descarbonylethoxyloratadine after storage at a temperature of from about 25° C. to about 40° C. and a relative humidity of about 60% to about 75% for at least 1 month. Also provided are stable pharmaceutical compositions for oral administration comprising an anti-allergic effective amount of descarbonylethoxy-loratadine or a pharmaceutically acceptable salt thereof, and a pharmaceutically acceptable filler wherein the composition is substantially free of a pharmaceutically acceptable basic salt and a lactose-based excipient and wherein the composition contains less than about 1% by weight of N-formyl descarbonylethoxyloratadine after storage at a temperature of from about 25° C. to about 40° C. and a relative humidity of about 60% to about 75% for at least 1 month.

### PRIORITY

This application claims the benefit under 35 U.S.C. §119 to U.S. Provisional Application No. 60/801,683, filed on May 19, 2006, and entitled “STABLE PHARMACEUTICAL COMPOSITION CONTAINING DESLORATADINE” and to Indian Provisional Application No. 1320/MUM/2005, filed on Oct. 20, 2005, and entitled “PHARMACEUTICAL COMPOSITION COMPRISING DESLORATADINE”, the contents of each of which are incorporated by reference herein.

### BACKGROUND OF THE INVENTION

1. Technical Field

The present invention generally relates to a pharmaceutical composition containing at least desloratadine.

2. Description of the Related Art

Desloratadine, also known as 8-chloro-6,11-dihydro-11-(4-piperidinylidene)-5H-benzo[5,6]cyclohepta[1,2-b]pyridine (“DCL”), may be represented by Formula I.

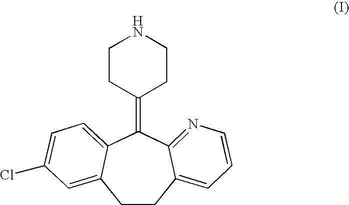

Desloratadine is a long-acting tricyclic histamine antagonist with selective H₁-receptor histamine antagonist activity. Desloratadine is indicated for the relief of the nasal and non-nasal symptoms of seasonal allergic rhinitis. Desloratadine is sold under the tradename Clarinex®. See, e.g., The Merck Index, Thirteenth Edition, 2001, p. 514, monograph 2939; and Physician's Desk Reference, “Clarinex,” 59th Edition, pp. 3021-3023 (2005).

U.S. Pat. No. 6,100,274 (“the '274 patent”) discloses that desloratadine discolors and decomposes due to a very minute amount of a degradation product caused by the presence of a wide variety of excipients commonly used in oral formulations. The excipients disclosed as unsuitable were acidic excipients including, but not limited to, stearic acid, povidone, and crospovidone, and other acidic excipients having a pH in water less than 7, preferably in the range of about 3 to 5 as well as other excipients such as lactose, lactose monohydrate, sodium benzoate, and Glyceryl Behenate NF sold under the tradename of Compritol® 888. In order to solve the problem of discoloration, the '274 patent discloses a pharmaceutical composition containing an anti-allergic effective amount of desloratadine in a pharmaceutically acceptable carrier medium containing a desloratadine-protective amount of a pharmaceutically acceptable basic salt.

It would be desirable to provide improved stable pharmaceutical compositions for oral administration containing an anti-allergic effective amount of descarbonylethoxy-loratadine or a pharmaceutically acceptable salt thereof.

### SUMMARY OF THE INVENTION

In accordance with one embodiment of the present invention, a stable pharmaceutical composition for oral administration is provided comprising an anti-allergic effective amount of descarbonylethoxy-loratadine or a pharmaceutically acceptable salt thereof, a lactose-based excipient and a pharmaceutically acceptable filler wherein the composition is substantially free of a pharmaceutically acceptable basic salt and wherein the composition contains less than about 1% by weight of N-formyl descarbonylethoxyloratadine after storage at about 25° C. and about 60% relative humidity for at least 1 month.

In accordance with a second embodiment of the present invention, a stable pharmaceutical composition for oral administration is provided comprising an anti-allergic effective amount of descarbonylethoxy-loratadine or a pharmaceutically acceptable salt thereof, a lactose-based excipient and a pharmaceutically acceptable filler wherein the composition is substantially free of a pharmaceutically acceptable basic salt and wherein the composition contains less than about 1% by weight of N-formyldescarbonylethoxyloratadine after storage at about 30° C. and about 65% relative humidity for at least 1 month.

In accordance with a third embodiment of the present invention, a stable pharmaceutical composition for oral administration is provided comprising an anti-allergic effective amount of descarbonylethoxy-loratadine or a pharmaceutically acceptable salt thereof, a lactose-based excipient and a pharmaceutically acceptable filler wherein the composition is substantially free of a pharmaceutically acceptable basic salt and wherein the composition contains less than about 1% by weight of N-formyldescarbonylethoxyloratadine after storage at about 40° C. and about 75% relative humidity for at least 1 month.

In accordance with a fourth embodiment of the present invention, a stable pharmaceutical composition for oral administration is provided comprising an anti-allergic effective amount of descarbonylethoxy-loratadine or a pharmaceutically acceptable salt thereof, and a pharmaceutically acceptable filler wherein the composition is substantially free of a pharmaceutically acceptable basic salt and a lactose-based excipient and wherein the composition contains less than about 1% by weight of N-formyl descarbonylethoxyloratadine after storage at about 25° C. and about 60% relative humidity for at least 1 month.

In accordance with a fifth embodiment of the present invention, a stable pharmaceutical composition for oral administration is provided comprising an anti-allergic effective amount of descarbonylethoxy-loratadine or a pharmaceutically acceptable salt thereof, and a pharmaceutically acceptable filler wherein the composition is substantially free of a pharmaceutically acceptable basic salt and a lactose-based excipient and wherein the composition contains less than about 1% by weight of N-formyldescarbonylethoxyloratadine after storage at about 30° C. and about 65% relative humidity for at least 1 month.

In accordance with a sixth embodiment of the present invention, a stable pharmaceutical composition for oral administration is provided comprising an anti-allergic effective amount of descarbonylethoxy-loratadine or a pharmaceutically acceptable salt thereof, and a pharmaceutically acceptable filler and a lactose-based excipient and wherein the composition is substantially free of a pharmaceutically acceptable basic salt and wherein the composition contains less than about 1% by weight of N-formyldescarbonylethoxyloratadine after storage at about 40° C. and about 75% relative humidity for at least 1 month.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

### DETAILED DESCRIPTION OF THE PREFERRED EMBODIMENTS

The present invention is directed to stable pharmaceutical compositions for oral administration containing an anti-allergic effective amount of descarbonylethoxy-loratadine or a pharmaceutically acceptable salt thereof. In one embodiment, the pharmaceutical composition contains an anti-allergic effective amount of descarbonylethoxy-loratadine or a pharmaceutically acceptable salt thereof, a lactose-based excipient and a pharmaceutically acceptable filler wherein the composition is substantially free of a pharmaceutically acceptable basic salt and wherein the composition contains less than about 1% by weight of N-formyldescarbonylethoxyloratadine after storage at a temperature ranging from 25° C. to about 40° C. and a relative humidity of about 60% to about 75% for at least 1 month. In another embodiment, the pharmaceutical composition contains an anti-allergic effective amount of descarbonylethoxy-loratadine or a pharmaceutically acceptable salt thereof, and a pharmaceutically acceptable filler wherein the composition is substantially free of a pharmaceutically acceptable basic salt and a lactose-based excipient and wherein the composition contains less than about 1% by weight of N-formyldescarbonylethoxyloratadine after storage at a temperature ranging from about 25° C. to about 40° C. and a relative humidity of about 60% to about 75% for at least 1 month.

The term “pharmaceutically acceptable basic salts”, which the pharmaceutical compositions of the present invention are substantially free of, includes such basic salts as the calcium, magnesium or aluminum salt, or mixtures thereof, including, but not limited to carbonates, phosphates, silicates and sulfates of calcium, magnesium and aluminum. Examples of such pharmaceutically acceptable basic salts include calcium sulfate anhydrous, hydrates of calcium sulfate, such as calcium sulfate dihydrate, magnesium sulfate anhydrous, hydrates of magnesium sulfate, dibasic calcium phosphate, dibasic calcium phosphate anhydrous, tribasic calcium phosphate, calcium silicate, magnesium silicate, magnesium trisilicate, aluminum silicate, and magnesium aluminum silicate.

The desloratadine used in the present invention may be obtained from loratadine, by hydrolysis of the carbamate, preferably under basic conditions. Loratadine itself may be prepared from N-methyl desloratadine by removing N-methyl group of N-methyl desloratadine by formation of the carbamate through reaction with a haloformate. The haloformate used may be an alkyl or aryl formate, with optional halogen substituted at first and/or second position of the formate, i.e., 2-chloroethyl-chloroformate. The carbamate may be prepared in an anhydrous C₅-C₁₂ hydrocarbon, such as toluene. When N-methyl desloratadine is used as a stating material, loratadine may or may not be isolated in preparation of desloratadine. The removal of the carbamate group of loratadine may be carried out with a base at elevated temperature. A preferred temperature is reflux temperature. A preferred base is an alkali metal or alkaline earth metal base such as potassium or sodium hydroxide. A preferred solvent is a C₁-C₄ alcohol such as 2-propanol. If desired, the desloratadine used herein may be in a polymorphic form, e.g., Form I and/or II, or mixtures thereof which can be prepared by techniques known in the art.

The lactose-based excipient for use herein includes, but is not limited to, lactose, anhydrous lactose, lactose monohydrate and the like and combinations thereof.

A suitable filler for use in the pharmaceutical composition of the present invention can be a water-insoluble starch product. Useful starch products include, but are not limited to, modified starches, agglomerated starches, granulated starches, pregelatinized starches and the like and combinations thereof. Examples of such starch products include Starch 1500, potato starch, corn starch, wheat starch, and the like and mixtures thereof. In one embodiment, suitable fillers for use herein include, but are not limited to, microcrystalline cellulose, dicalcium phosphate, mannitol and the like and combination thereof.

The stable pharmaceutical compositions of the present invention can be formulated into a solid or liquid dosage form. Solid form compositions include powders, tablets, dispersible granules, capsules, cachets, and suppositories. In the case of powders, in one embodiment, the lactose-based excipient is a finely divided solid which is in admixture with the finely divided active ingredient and the filler. In the case of a tablet, in one embodiment, the active ingredient is mixed with a lactose-based excipient and filler having the necessary binding properties in suitable proportions and compacted in the shape and size desired. The anti-allergic effective amount of DCL in the pharmaceutical compositions of this invention, e.g., powders and tablets, is from about 0.5 to about 15 weight percent, preferably about 0.5 to about 10 weight percent, and more preferably about 1 to about 10 weight percent, based on the total weight of the composition.

The anti-allergic effective amount of descarbonylethoxyloratadine for oral administration varies from about 1 to about 50 mg/day, preferably about 2.5 to about 20 mg/day and more preferably about 5 to about 10 mg/day in single or divided doses. The most preferred amount is about 5 mg, once a day. Of course the precise dosage and dosage regimen may be varied depending upon the requirements of the patients (e.g., his or her sex, age, etc.) as well as the severity of the allergic condition being treated. Determination of the proper dosage and dosage regimen for a particular patient will be within the skill of the attending clinician.

The major decomposition product of DCL found in the pharmaceutical compositions of the present invention is N-formyl descarbonylethoxyloratadine. The pharmaceutical compositions of the present invention contain less than about 1% by weight at periods up to at least 1 month. Preferably, the pharmaceutical compositions of the present invention contain less than about 1% by weight, preferably less than about 0.5% by weight, more preferably less than about 0.1% by weight, and most preferably less than about 0.05% by weight of N-formyl descarbonylethoxyloratadine when such compositions were stored at about 25° C. and about 60% relative humidity (RH) for at least 1 month or at about 30° C. and about 65% RH for at least 1 month or at about 40° C. and about 75% RH for at least 1 month.

The following examples are provided to enable one skilled in the art to practice the invention and are merely illustrative of the invention. The examples should not be read as limiting the scope of the invention as defined in the claims.

### EXAMPLE 1

The formulation of this example is set forth in Table 1.

TABLE 1

| | |
|---|---|
| Ingredients | Mg/tablet |

I. Dry Mix

| | |
|---|---|
| Microcrystalline cellulose | 35.00 |
| Corn starch | 31.00 |

II. Binder Solution

| | |
|---|---|
| Hydroxypropyl methyl | 2.00 |
| cellulose | |
| Purified water | Q.S |

III. Lubrication

| | |
|---|---|
| Base Granules | 68.00 |
| Desloratadine | 5.00 |
| Corn Starch | 9.55 |
| Microcrystalline cellulose | 5.00 |
| Talc | 2.00 |
| Magnesium stearate | 0.45 |

A dry mix was prepared by sifting starch and microcrystalline cellulose through a 40# American Society for Testing and Materials Standards (ASTM) sieve and further mixed uniformly. A binder solution was prepared by dissolving hydroxypropylmethyl cellulose in purified water. The dry mix was then loaded and blended in a rapid mixer granulator. The binder solution prepared was used to granulate the sifted dry mix of microcrystalline cellulose and starch in a high shear mixer granulator. The granules thus obtained were subjected to drying in a fluid bed drier. Next, the dried granules were sifted through a 30# ASTM sieve and blended with desloratadine, starch, microcrystalline cellulose, and talc. The blended granules were lubricated with magnesium stearate and then compressed into tablets. The tablets were kept uncoated.

The uncoated tablets prepared in accordance with Example 1, were subjected to stability studies by storing the samples in a high density polyethylene (HDPE) container at (1) 25° C. and 60% RH, (2) 30° C. and 65% RH and (3) 40° C. and 75% RH. The % of degradation, e.g., N-formyl desloratadine, was determined after each month until the completion of three months. Based on the impurity profile presented in table 2, the composition described in example 1 complies with the guidelines led by the U.S. Food and Drug Administration (FDA), for impurities in a drug substance. The results of the studies are set forth in Table 2.

TABLE 2

| | | | % N-Formyl | % | % Total |
|---|---|---|---|---|---|
| Stability | Stability | % | desloratadine | Single | Impu- |
| Period | Condition | Assay | Impurity | Max. | rity |
| Initial | — | 103.2 | *Not | 0.05 | 0.08 |
| | | | Determined | | |
| | 25° C./60% RH | 103.5 | 0.021 | 0.07 | 0.16 |
| 1 Month | 30° C./65% RH | 103.1 | 0.024 | 0.07 | 0.17 |
| | 40° C./75% RH | 103.9 | 0.053 | 0.07 | 0.20 |
| | 25° C./60% RH | — | — | — | — |
| 2 Month | 30° C./65% RH | — | — | — | — |
| | 40° C./75% RH | 102.4 | 0.023 | 0.04 | 0.17 |
| | 25° C./60% RH | 101.8 | 0.024 | 0.06 | 0.17 |
| 3 Month | 30° C./65% RH | — | — | — | — |
| | 40° C./75% RH | 104.1 | 0.118 | 0.22 | 0.50 |

As the data show, the pharmaceutical compositions of present invention, when subjected to a temperature of 40° C. and 75% RH for three month, yielded less than 0.2% of the N-formyl desloratadine impurity.

### EXAMPLE 2

The formulation of this example is set forth in Table 3.

TABLE 3

| | |
|---|---|
| Ingredients | Mg/tablet |

I. Dry Mix

| | |
|---|---|
| Desloratadine | 5.00 |
| Corn starch | 20.55 |
| Lactose | 55.00 |
| Pregelatinized starch | 2.00 |

II. Binder Solution

| | |
|---|---|
| Purified water | Q.S |

III. Lubrication

| | |
|---|---|
| Corn starch | 7.00 |
| Magnesium stearate | 0.45 |

Desloratadine and corn starch were co-sifted through a 60# ASTM sieve. Lactose monohydrate and pregelatinized starch were sifted through a through a 40# ASTM sieve and all of the sifted excipients were mixed in a rapid mixer granulator. Next, purified water was added to the granulate in a high shear mixer granulator. The granules thus obtained were then dried in a fluid bed dryer. The dried granules were sifted through a through a 30# ASTM sieve. The sifted granules were blended with starch and lubricated with magnesium stearate. The lubricated granules were compressed into tablets. The tablets were kept uncoated.

The uncoated tablets prepared in accordance with example 2, were subjected to stability studies by storing the samples in a HDPE container at (1) 25° C. and 60% RH, (2) 30° C. and 65% RH and (3) 40° C. and 75% RH. The % of degradation, e.g., N-formyl desloratadine, was determined after each month until the completion of three months. Based on the impurity profile presented in Table 2, the composition described in example 2 complies with the guidelines led by the U.S. Food and Drug Administration (FDA), for impurities in a drug substance. The results of the studies are set forth in Table 4.

TABLE 4

| | | | % N-Formyl | % | % Total |
|---|---|---|---|---|---|
| Stability | Stability | % | desloratadine | Single | Impu- |
| Period | Condition | Assay | Impurity | Max. | rity |
| Initial | — | 96.8 | *Not | 0.08 | 0.10 |
| | | | detected | | |
| | 25° C./60% RH | 100.2 | 0.012 | 0.06 | 0.17 |
| 1 Month | 30° C./65% RH | 99.3 | 0.010 | 0.06 | 0.17 |
| | 40° C./75% RH | 100.8 | 0.019 | 0.06 | 0.18 |
| | 25° C./60% RH | — | — | — | — |
| 2 Month | 30° C./65% RH | — | — | — | — |
| | 40° C./75% RH | 100.3 | 0.022 | 0.06 | 0.17 |
| | 25° C./60% RH | 99.9 | 0.011 | 0.06 | 0.14 |
| 3 Month | 30° C./65% RH | — | — | — | — |
| | 40° C./75% RH | 99.1 | 0.033 | 0.05 | 0.16 |

### As the data show, the pharmaceutical compositions of present invention, when subjected to a temperature of 40° C. and 75% RH for three months, yielded less than 0.2% of the N-formyl desloratadine impurity.

### EXAMPLE 3

The formulation of this example is set forth in Table 5.

TABLE 5

| | |
|---|---|
| Ingredients | Mg/tablet |

I. Dry Mix

| | |
|---|---|
| Corn starch (Purity 21A) | 5.00 |
| Mannitol 25 (Pearlitol) | 20.55 |
| Pregelatinized starch | 55.00 |
| (Pregel PA 5PH) | |

II. Binder Solution

| | |
|---|---|
| Purified water | Q.S |

III. Lubrication

| | |
|---|---|
| Base granules | 7.00 |
| Desloratadine | 5.00 |
| Corn starch (Purity 21A) | 0.45 |
| Magnesium stearate | 0.5 |

Mannitol, corn starch and pregelatinized starch were sifted through a 40# ASTM and mixed in a high shear mixer granulator. Next, purified water was added to the granulates in a high shear mixer granulator. The wet mass was milled through a suitable sieve to provide granules. The granules thus obtained were subjected to drying in a fluid bed drier. The dried granules were sifted through a 30# ASTM sieve. Desloratadine and starch were co-sifted through a 30# ASTM sieve. The base granules were then blended with the blend of desloratadine and starch and then lubricated with magnesium stearate. The lubricated blend was compressed into tablets. The compressed tablets were packed in HDPE containers.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A stable pharmaceutical composition for oral administration comprising an anti-allergic effective amount of descarbonylethoxy-loratadine or a pharmaceutically acceptable salt thereof, a lactose-based excipient and a pharmaceutically acceptable filler, wherein the composition is substantially free of a pharmaceutically acceptable basic salt and wherein the composition contains less than about 1% by weight of N-formyl descarbonylethoxyloratadine after storage at a temperature of from about 25° C. to about 40° C. and a relative humidity of about 60% to about 75% for at least 1 month.

2. The stable pharmaceutical composition of claim 1, wherein the composition contains less than about 1% by weight of N-formyldescarbonylethoxyloratadine after storage at about 25° C. and about 60% relative humidity for at least 1 month.

3. The stable pharmaceutical composition of claim 1, wherein the composition contains less than about 0.5% by weight of N-formyldescarbonylethoxyloratadine after storage at about 25° C. and about 60% relative humidity for at least 1 month.

4. The stable pharmaceutical composition of claim 1, wherein the composition contains less than about 1% by weight of N-formyldescarbonylethoxyloratadine after storage at about 30° C. and about 65% relative humidity for at least 1 month.

5. The stable pharmaceutical composition of claim 1, wherein the composition contains less than about 0.5% by weight of N-formyldescarbonylethoxyloratadine after storage at about 30° C. and about 65% relative humidity for at least 1 month.

6. The stable pharmaceutical composition of claim 1, wherein the composition contains less than about 1% by weight of N-formyldescarbonylethoxyloratadine after storage at about 40° C. and about 75% relative humidity for at least 1 month.

7. The stable pharmaceutical composition of claim 1, wherein the composition contains less than about 0.5% by weight of N-formyldescarbonylethoxyloratadine after storage at about 40° C. and about 75% relative humidity for at least 1 month.

8. The stable pharmaceutical composition of claim 1, wherein the lactose-based excipient is selected from the group consisting of lactose, anhydrous lactose, lactose monohydrate and combinations thereof.

9. The stable pharmaceutical composition of claim 1, wherein the filler is a water-insoluble starch product.

10. The stable pharmaceutical composition of claim 1, wherein the filler is selected from the group consisting of modified starches, agglomerated starches, granulated starches, pregelatinized starches and combinations thereof.

11. The stable pharmaceutical composition of claim 1, wherein the filler is selected from the group consisting of Starch 1500, potato starch, corn starch, wheat starch, and mixtures thereof.

12. The stable pharmaceutical composition of claim 1, in a solid dosage form.

13. A stable pharmaceutical composition for oral administration comprising an anti-allergic effective amount of descarbonylethoxy-loratadine or a pharmaceutically acceptable salt thereof, and a pharmaceutically acceptable filler wherein the composition is substantially free of a pharmaceutically acceptable basic salt and a lactose-based excipient and wherein the composition contains less than about 1% by weight of N-formyl descarbonylethoxyloratadine after storage at a temperature of from about 25° C. to about 40° C. and a relative humidity of about 60% to about 75% for at least 1 month.

14. The stable pharmaceutical composition of claim 13, wherein the composition contains less than about 0.5% by weight of N-formyldescarbonylethoxyloratadine after storage at about 25° C. and about 60% relative humidity for at least 1 month.

15. The stable pharmaceutical composition of claim 13, wherein the composition contains less than about 0.1% by weight of N-formyldescarbonylethoxyloratadine after storage at about 25° C. and about 60% relative humidity for at least 1 month.

16. The stable pharmaceutical composition of claim 13, wherein the composition contains less than about 0.5% by weight of N-formyldescarbonylethoxyloratadine after storage at about 30° C. and about 65% relative humidity for at least 1 month.

17. The stable pharmaceutical composition of claim 13, wherein the composition contains less than about 0.1% by weight of N-formyldescarbonylethoxyloratadine after storage at about 30° C. and about 65% relative humidity for at least 1 month.

18. The stable pharmaceutical composition of claim 13, wherein the composition contains less than about 0.5% by weight of N-formyldescarbonylethoxyloratadine after storage at about 40° C. and about 75% relative humidity for at least 1 month.

19. The stable pharmaceutical composition of claim 13, wherein the composition contains less than about 0.1% by weight of N-formyldescarbonylethoxyloratadine after storage at about 40° C. and about 75% relative humidity for at least 1 month.

20. The stable pharmaceutical composition of claim 13, in a solid dosage form.

21. The stable pharmaceutical composition of claim 13, in a form of a tablet, a caplet, a capsule, a suspension tablet, a troche, or a powder.

22. The stable pharmaceutical composition of claim 13, further comprising one or more pharmaceutically acceptable excipients.

23. The stable pharmaceutical composition of claim 13, further comprising a lubricant.

24. The stable pharmaceutical composition of claim 13, wherein the filler is selected from the group consisting of microcrystalline cellulose, dicalcium phosphate, mannitol and combinations thereof.
